(19) United States
(12) Patent Application Publication
(10) Pub. No.: US 2015/0346040 A1
(43) Pub. Date: Dec. 3, 2015

(54) Grain Monitoring Device

(71) Applicant: Gary Brandon Stephens, Cherry Valley, AR (US)

(72) Inventor: Gary Brandon Stephens, Cherry Valley, AR (US)

(21) Appl. No.: 14/730,054

(22) Filed: Jun. 3, 2015

Related U.S. Application Data
(60) Provisional application No. 62/006,979, filed on Jun. 3, 2014.

(51) Int. Cl. G01K 13/00; G01N 33/10; G01N 25/56

(52) U.S. Cl. CPC G01K 13/00; G01N 25/56; G01N 33/10

(57) ABSTRACT

A device for monitoring and synthesizing grain moisture and temperature within a grain storage facility is disclosed. The device can wirelessly transmit the collected data to smart mobile devices thus eliminating the need for an individual to physically climb into the storage facility and monitor the levels.

### CROSS-REFERENCES TO RELATED APPLICATIONS

This application claims the benefit of U.S. Provisional Application No. 62/006,979, filed Jun. 3, 2014, which is incorporated herein by reference in its entirety.

### FIELD OF THE INVENTION

The present invention is generally directed toward a device for monitoring and transmitting grain moisture and temperature levels within a grain storage facility.

### BACKGROUND OF THE INVENTION

Grain monitoring devices are used to monitor grain temperature and moisture levels within a grain storage facility. These levels must be monitored because the grain can spoil and be wasted if stored at the wrong conditions. The devices that are currently available require individuals to climb inside the grain storage facility and physically monitor the temperature and moisture levels. It is common for individuals become victims of grain entrapment and to suffocate while working inside a grain storage facility making measuring the temperature and moisture levels a costly and time consuming activity.

The devices known in the art have sensors and communicate data to a central data storage system via a cellular network or long range radio frequency data transmission. Alerts and alarms can be transmitted electronically to users via email, SMS text, or automated phone messages. Another such device comprises a system for remotely reporting the level of grain loaded into a grain trailer, wherein periodic samples can be drawn of the flowing material and wirelessly transmitted form the sample device to a computer used with sensor strips. These devices, however, do not utilize a sensor with a transmitter that can monitor and synthesize the moisture and temperature of the grain and wirelessly transmit the collected data to smart mobile devices.

### SUMMARY OF THE INVENTION

The presently disclosed device is a grain monitoring system designed to monitor temperature and moisture levels of grain within a grain storage facility and transmit the collected data to a smart mobile device. The device comprises an interior sensor contained within a rod-shaped enclosure. The sensor reads, records, synthesizes, and reports the grain temperature and moisture levels at two different sensing depths multiple times throughout the day. The radio transmitter on the opposite end of the enclosure transmits a signal to an exterior base which then sends radio transmissions to a Wi-fi hub. Application software then allows wireless reporting to smart mobile devices, including but not limited to, phones and pads. Further, the network can be upgraded to include cloud reporting and cellular messaging.

### BRIEF DESCRIPTION OF THE DRAWINGS

Further advantages of the invention will become apparent by reference to the detailed description of preferred embodiments when considered in conjunction with the drawings:

FIG. 1 depicts a perspective view of the interior sensor.

FIG. 2 depicts another perspective view of the interior sensor.

FIG. 3 depicts a perspective view of the first transmitter.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The invention claimed herein allows an individual to monitor the temperature and moisture levels of grain in a grain storage facility without having to physically climb into the grain storage facility to monitor the levels. Furthermore, it does not require transmission to a computer before wirelessly transmitting the data to an end user.

The disclosed device comprises an interior sensor 1, an exterior base, and a Wi-fi hub for transmitting the data collected from the interior sensor 1

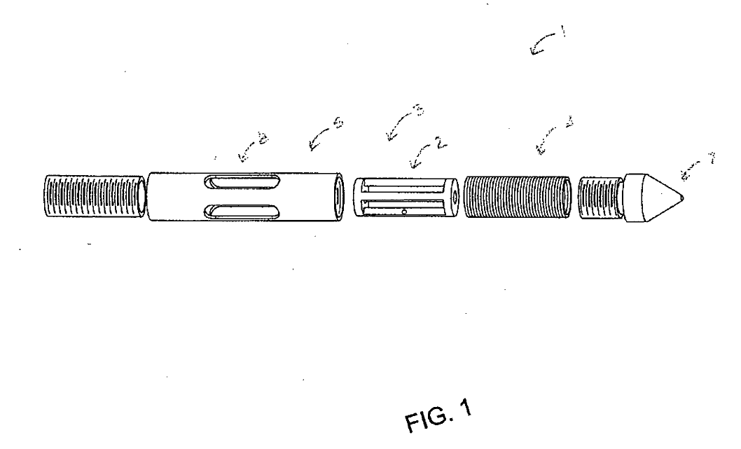

The interior sensor 1 further comprises a battery, electronic moisture sensing modules 2, and a first radio transmitter. The interior programmable sensor is simple to use and operate. As shown in FIG. 1, the interior sensor 1 is contained within a rod-shaped enclosure 3 that is inserted into the grain within the storage facility. Said interior sensor 1 reads, records, reports and synthesizes grain temperature moisture content. Further, the claimed invention allows a user of the device to choose between two different sensing depths selectable from one, two or three meters. The interior sensor 1 can record the temperature and moisture levels multiple times per day and report minimum, maximum and average levels for each day.

The preferred embodiment of the rod-shaped enclosure 3 comprises a stainless steel rod with properties for increased durability and corrosion resistance.

In another embodiment, the interior sensor 1 measures and reports temperature and moisture levels of the grain with 2% accuracy and repeatability as high as 0.5% (preliminary).

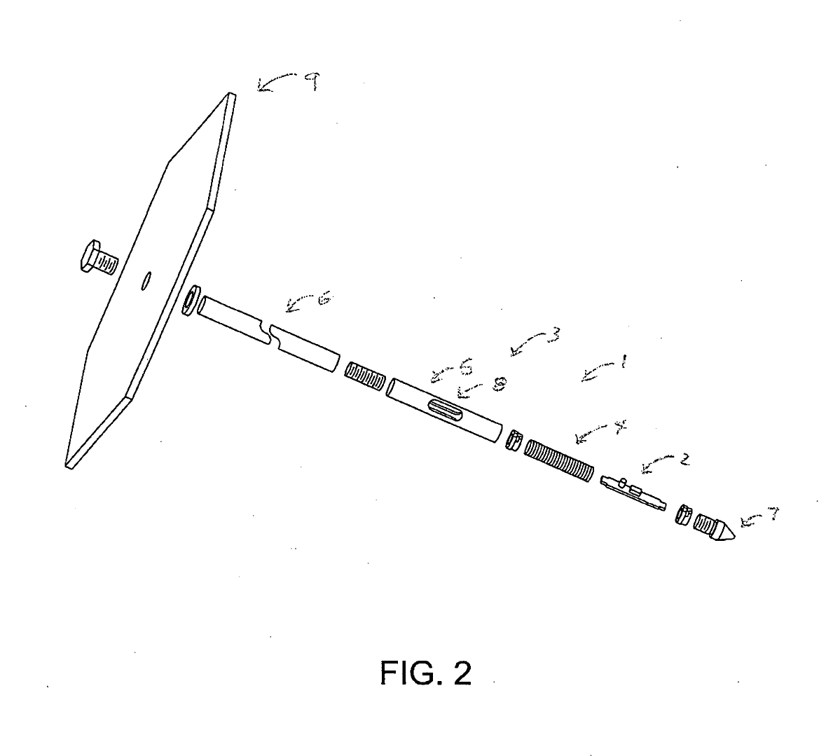

As shown in FIG. 2, additional features of the interior sensor 1 include threaded couplings 4, a sensor housing 5, and an abbreviated tube 6.

In another potential embodiment, the interior sensor 1 includes a point 7 at the first end wherein said end is inserted into the grain storage facility to monitor the moisture and temperature of the grain. Hollow threaded couplings 4 allow electrical wires to pass through the rod-shaped enclosure 3. The cylindrical sensor housing 5 contains gaps 8 to allow moisture into said rod. Further, an abbreviated tube 6 allows for the length of the rod to vary.

Other potential embodiments of the abbreviated tube 6 may include lengths of three, six, nine, or twelve feet in length.

In another embodiment, the interior sensor 1 also includes a stop disc 9 is located at the second end of the rod-shaped enclosure 2 which is perpendicular to said rod. When the rod-shaped enclosure 3 is inserted into the grain, the stop disc 9 disables the rod-shaped enclosure 3 from being inserted any farther into the grain.

Preferably, the interior sensor battery has greater than a one year battery life with battery condition reporting.

The first radio transmitter transmits the collected data via radio frequency from an antennae to an exterior base located on the outside of the grain storage facility.

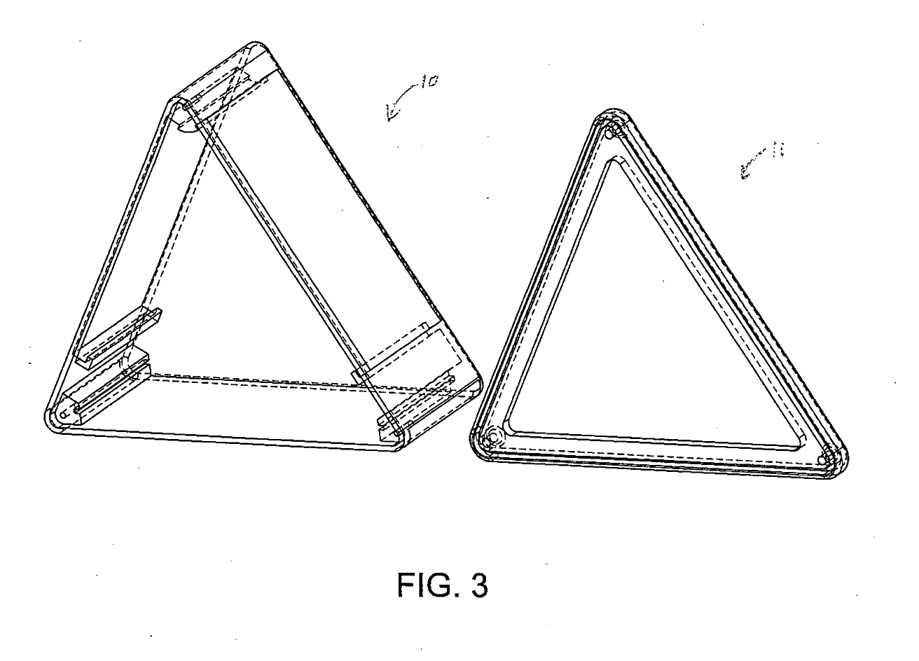

A potential embodiment of the first radio transmitter, it is housed in a tri-lobe case 10 with a cover 11 as shown in FIG. 3. The tri-lobe case 10 and cover 11 is located above the stop disc 9.

The exterior base comprising a battery, wireless receiver, and second radio transmitter. The data is transmitted via radio frequency from the exterior base to a Wi-fi hub. One storage facility out of numerous facilities within a seven mile line of sight radius contains a Wi-fi hub which then transmits the data wirelessly via application software to a smart mobile device, including without limitation, phones and pads.

A potential embodiment of the Wi-fi hub has a solar powered battery with a life of greater than one year.

In another potential embodiment of the Wi-fi hub, the data transmission is via cloud reporting. The connection to the interne may also be through a cellular connection.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

I claim:

1. A device for monitoring temperature and moisture of grain within a silo comprising:
a. An interior sensor contained within a rod-shaped enclosure;
b. An exterior base; and
c. a wifi hub;

2. The device of claim 1 wherein said rod is capable of separating into a plurality of pieces

3. The device of claim 1 wherein one end of said rod is attached to a stop disc which is perpendicular to said rod.

4. The device of claim 1 wherein said interior sensor further comprising:
a. A battery;
b. An electronic moisture sensing modules
c. First radio transmitter.

5. A exterior base further comprising
a. A battery;
b. Wireless receiver;
c. Second radio transmitter.

6. The wifi hub capable of receiving radio transmissions from said second radio transmitter.

7. The device of claim 6 wherein said network connection to the internet is broadband.

8. The device of claim 6 wherein said network connection to the interne is cellular data.

9. The device of claim 6 wherein said transmission is via cloud reporting.

10. The device of claim 6 wherein said transmission is controlled by software.
